(19) United States
(12) Patent Application Publication
(10) Pub. No.: US 2013/0224805 A1
(43) Pub. Date: Aug. 29, 2013

(54) System and Method for Converting Cellulosic Biomass into a Sugar Solution

(71) Applicants: Darrell M. Waite, Bangor, ME (US); Richard Arnold, Milford, ME (US); James St. Pierre, Milford, ME (US); Rino P. Dumont, Hermon, ME (US); James Alan Hargreaves, Old Town, ME (US); William H. Ceckler, Hancock, ME (US)

(72) Inventors: Darrell M. Waite, Bangor, ME (US); Richard Arnold, Milford, ME (US); James St. Pierre, Milford, ME (US); Rino P. Dumont, Hermon, ME (US); James Alan Hargreaves, Old Town, ME (US); William H. Ceckler, Hancock, ME (US)

(21) Appl. No.: 13/592,682

(22) Filed: Aug. 23, 2012

Related U.S. Application Data
(60) Provisional application No. 61/526,877, filed on Aug. 24, 2011.

(51) Int. Cl. C12P 19/02

(52) U.S. Cl. CPC C12P 19/02
USPC 435/105; 435/289.1; 435/297.1; 204/542; 204/627

(57) ABSTRACT

A process and apparatus for converting cellulosic biomass pulp into a sugar solution is provided. The process includes combining a quantity of cellulosic biomass pulp with a quantity of acid and a quantity of enzyme. The combined quantity of cellulosic biomass pulp, enzyme, and acid are placed in an enzymatic hydrolysis reactor having a predetermined temperature range and predetermined pH level, thereby producing a quantity of monomeric sugar solution.

### STATEMENT REGARDING FEDERALLY SPONSORED RESEARCH OR DEVELOPMENT

This invention was made in part with Government support under Contract No. DE-EE 0003364 awarded by U S Department of Energy. The Government may have certain rights in the invention.

### CROSS REFERENCE TO RELATED APPLICATION

This application claims benefit of U.S. Provisional Application Ser. No. 61/526,877, entitled, “System and Method for Converting Woody Biomass into Sugar” filed Aug. 24, 2011, the entire disclosure of which is incorporated herein by reference.

### FIELD OF THE DISCLOSURE

The present disclosure is generally related to biomass conversion systems and processes and more particularly is related to a system and process for converting cellulosic biomass materials into a sugar solution.

### BACKGROUND OF THE DISCLOSURE

The extraction of various substances, such as raw liquid extract, from a biomass material is a common and necessary process when making paper or other cellulose-based materials. Wood and other cellulosic biomass naturally contain substances useful for processing into bio-fuel and other products. Cellulosic biomass is one of the largest growing crops on the globe, as measured by mass of the sugar per acre produced, and woody biomass is a sustainable renewable resource that is not designated as a food crop. However, while wood and Cellulosic biomass are regularly processed for making paper or other cellulose-based materials, it has never been efficiently processed into bio-fuel products with commercial success. Particularly, conventional systems are often unable to efficiently remove certain inhibitors from the biomass material, and do so in a cost-effective manner. Furthermore, conventional systems may be unable to monomerize oligimeric sugars contained within the processed cellulosic biomass material and, as a result, unable to produce either a solid or a liquid solution of the sugars.

Thus, an unaddressed need exists in the industry to provide a system and method for converting wood and other cellulosic biomass into sugar.

### SUMMARY OF THE DISCLOSURE

Embodiments of the present disclosure provide an apparatus and process for converting cellulosic biomass pulp suspension into a sugar solution. Briefly described, one embodiment of such a method, among others, can be broadly summarized by the following steps: combining a quantity of cellulosic biomass pulp suspension with a quantity of acid and a quantity of enzyme; and placing the combined quantity of cellulosic biomass pulp suspension, enzyme, and acid in an enzymatic hydrolysis reactor having a predetermined temperature range and predetermined pH level, thereby producing a quantity of monomeric sugar solution.

The present disclosure can also be viewed as providing an apparatus for converting cellulosic biomass pulp into sugar. Briefly described, one embodiment of such an apparatus, among others, can be implemented as follows: A first mixer is provided for mixing cellulosic biomass pulp and a quantity of acid. A second mixer fed from the first mixer mixes the cellulosic biomass pulp and acid with a quantity of enzyme. An enzymatic hydrolysis reactor receives the mixed cellulosic biomass pulp, acid, and enzyme, wherein the enzymatic hydrolysis reactor has a predetermined temperature range and a predetermined pH level, and wherein the enzymatic hydrolysis reactor outputs a quantity of monomeric sugar solution.

The present disclosure can also be viewed as providing processes for converting cellulose rich biomass pulp into a C6 rich sugar solution. In this regard, one embodiment of such a method, among others, can be broadly summarized by the following steps: combining a quantity of cellulose rich biomass pulp with a quantity of acid and a quantity of enzyme; and placing the combined quantity of cellulose rich biomass pulp, enzyme, and acid in an enzymatic hydrolysis reactor having a predetermined temperature range and predetermined pH level, thereby producing a quantity of monomeric C6 rich sugar solution.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic illustration of a system for converting cellulosic biomass pulp suspension into a sugar solution, in accordance with a first exemplary embodiment of the present disclosure.

FIG. 2 is a schematic illustration of a system for converting cellulosic biomass into a sugar solution, in accordance with a second exemplary embodiment of the present disclosure.

FIG. 3 is a schematic illustration of a system for converting cellulose rich biomass pulp into a C6 rich sugar solution, in accordance with a third exemplary embodiment of the present disclosure.

FIG. 4 is a schematic illustration of a system for converting cellulose rich biomass into a C6 sugar rich solution, in accordance with a fourth exemplary embodiment of the present disclosure.

FIG. 5 is a flowchart illustrating a process for converting cellulosic biomass pulp into a sugar solution, in accordance with the first exemplary embodiment of the present disclosure.

FIG. 6 is a flowchart illustrating a process for converting cellulosic biomass pulp into a sugar solution, in accordance with the second exemplary embodiment of the present disclosure.

FIG. 7 is a flowchart illustrating a process for converting cellulosic rich biomass pulp into a C6 rich sugar solution, in accordance with the third exemplary embodiment of the present disclosure.

FIG. 8 is a flowchart illustrating a process for converting cellulosic rich biomass pulp into a C6 rich sugar solution, in accordance with a fourth exemplary embodiment of the present disclosure.

### DETAILED DESCRIPTION

In order to understand the following disclosures of systems and processes it is necessary to understand the following definitions of materials used in the processes:
1. Brown stock is an aqueous suspension of un-bleached cellulose and hemicelluloses fibers, derived from woody biomass by processing it through any of a variety of pulping processes, such as but not limited to: kraft, soda, sulfite, neutral sulfite, and mechanical pulping processes. For the purposes of this disclosure brown stock will be referred to as cellulosic biomass pulp suspension.
2. There are many other sources of cellulosic biomass other than woody biomass, such as: tobacco plant stalks, corn stalks and stovers, grasses and other plants which can be converted to an aqueous suspension of cellulose and hemicelluloses fibers by processing them through the same pulping processes as used for woody biomass. For the purpose of this disclosure aqueous suspensions of cellulose and hemicelluloses fibers produced from non-woody biomass will also be referred to as cellulosic biomass pulp suspensions.

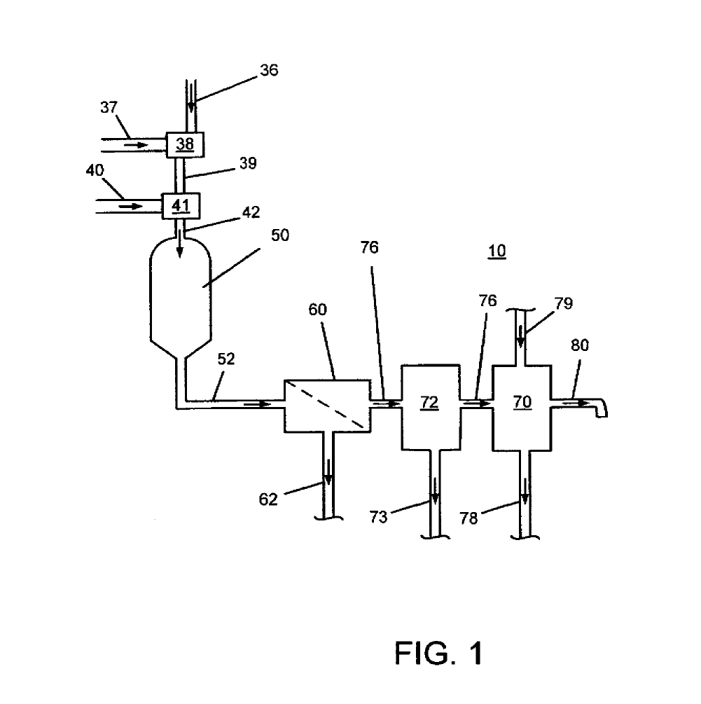

FIG. 1 is a schematic illustration of a system for converting cellulosic biomass pulp suspension into a sugar solution, in accordance with a first exemplary embodiment of the present disclosure. The system 10 for producing a sugar solution, which may be referred to herein as ‘the system 10’, may include any of the features, components, or functions disclosed with respect to any of the other embodiments within this disclosure. The system 10 includes a cellulosic biomass pulp suspension within conduit 36 which is combined with sulfuric acid via conduit 37 and then placed within a mixer 38. The mixed cellulosic biomass pulp suspension and sulfuric acid are transported out of the mixer 38 via conduit 39 and combined with a quantity of enzyme that are transferred via conduit 40 into mixer 41 and then transferred by conduit 42 to an enzymatic hydrolysis reactor 50. The cellulosic biomass pulp suspension, sulfuric acid, and the quantity of enzyme are held within the enzymatic hydrolysis reactor 50 for a period of time, preferably between 12-100 hours although other period of time may be acceptable. While in the enzymatic hydrolysis reactor 50, the cellulosic biomass pulp suspension, sulfuric acid, and the quantity of enzyme are subjected to an approximate temperature between 35-70° C. at an approximate pH of 3.0-7.5. The cellulosic biomass pulp suspension, sulfuric acid, and the quantity of enzyme are output as a quantity of monomeric sugar solution via output line 52, wherein the quantity of monomeric sugar solution may have a total sugar concentration of approximately between 5% and 25%.

A variety of different devices and components to the system I 0 may be used at various stages. Commonly, after the enzymatic hydrolysis reactor 50, the quantity of monomeric sugar solution is transferred to a filter 60 to remove unprocessed pulp and lignin and spent cooking chemicals from the quantity of monomeric sugar solution. The filter may be any type of filtering device or washing device that can separate suspended solids from liquids, such as but not limited to filter press, centrifuge or ultra-filtration system. Any residual solids may be output from the filter 60 via output 62. The filtered quantity of monomeric sugar solution may then be transferred into a water removal device 72, such as an evaporator, via line 76, which may remove a quantity of water via line 73, to concentrate the filtered quantity of monomeric sugar to approximately 100-800 g/l. The concentrated filtered quantity of monomeric sugar may then be processed within a electrodialysis unit 70, connected to the water removal device 72 via line 76 to remove salts, acids, heavy metals, and lignin which are removed from the sugar solution by transferring them to a brine solution which enters the device via line 79 and leaves via line 78, thereby providing a sugar solution. The quantity of sugar solution may then be output via line 80 as a product of the system 10.

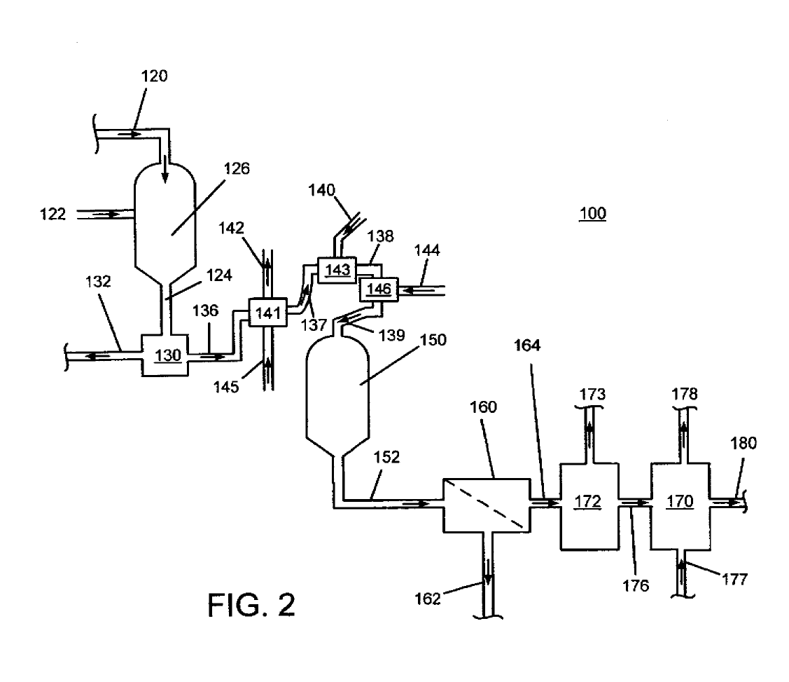

FIG. 2 is a schematic illustration of a system 100 for converting cellulosic biomass into a sugar solution, in accordance with a second exemplary embodiment of the present disclosure. The system 100 for converting cellulosic biomass into sugar, hereinafter simply referred to as the ‘system’ 100, may be used to extract a sugar solution from a cellulosic biomass feedstock, and may include any of the features, components, or functions disclosed with respect to any of the other embodiments within this disclosure.

As is shown in FIG. 2, an input line 120 may input a quantity of cellulosic biomass into the system 100. The quantity of cellulosic biomass may be transported into a vessel 126, which may be a traditional chemical pulp-cooking device, which utilizes heat and chemicals to separate a quantity of lignin from a quantity of fiber within the quantity of biomass. A quantity of fresh cooking liquor may also be input into the vessel 126 via line 122. The result is a quantity of cellulosic biomass pulp suspension, created from the quantity of cellulosic biomass, which leaves the vessel 126 via an outline line 124.

It is noted that the term ‘line’ herein may refer to any type of conduit, pipe, or substance transportation device. It is further noted that the arrangement of the components of the system 100 may be altered and adjusted as needed. This may include, for example, combining various substances within mixing devices or directly within conduits, transferring unconverted or unused materials back into the system 100 for additional use, and/or inserting additional components to further refine the substances processed within the system 100.

The process within the chemical pulp-cooking device may include any type of biomass pulping process, including kraft, sulfite, soda and near neutral sulfite. For example, in the kraft process, the woody biomass is treated with a mixture of sodium hydroxide, sodium carbonate, and sodium sulfide, known as ‘white liquor’ to break the bonds that link the lignin to the cellulose. Any of the various chemical processes used may have specific parameters and requirements, any of which may be incorporated into the system 100 without reservation. It should also be noted that mechanical or thermal mechanical pulping processes may be used.

The quantity of cellulosic biomass pulp suspension may be characterized as a material that is approximately 60%-98% cellulose. Other quantities of the cellulose within the quantity of cellulosic biomass may also be within the scope of the present disclosure. The remainder of the quantity of cellulosic biomass pulp, i.e., 2%-40%, may be primarily a hemi-cellulose material. As is described further herein, the quantity of cellulose material may be processed into glucose or other C6 sugars, whereas any portion of the quantity of hemi-cellulose material may be processed into xylose or other C5 sugars.

Filter 130 may filter the quantity of cellulosic biomass pulp to produce a suspension of cellulosic biomass pulp and a solution of spent cooking liquor which contains a quantity of the lignin which enters the process with the biomass and is known as black liquor. A portion of the cellulosic biomass suspension may be transferred to the next processing step through line 136. The black liquor or other spent cooking liquor may be disposed of or processed accordingly, such as via disposal line 132. The quantity of cellulosic biomass pulp suspension is transferred by line 136 to a pulp washer 141 which intakes water via line 145 and outputs dilute spent cooking liquor via line 142. The cellulosic biomass pulp suspension enters mixer 143 via line 137 where acid is added via line 140; the output continues through line 138 to mixer 146 where enzyme is added by line 144. The cellulosic biomass pulp, acid and enzyme suspension then passes to the enzyme hydrolysis reactor 150 through line 139. The enzymatic hydrolysis reactor may be any vessel or structure that allows for digestion, processing, or a catalytic transformation of the cellulose, hemicellulose suspension and the acid and enzyme to produce the quantity of monomeric sugar solution. The enzymatic hydrolysis reactor 150 may have a predetermined temperature range and predetermined pH level to produce the quantity of monomeric sugar solution. For example, the enzymatic hydrolysis reactor 150 may have a temperature range of 35° C. to 70° C. and a pH level between 3.0 and 7.5, although other ranges are considered within the scope of the present disclosure. Additionally, the feed to the enzymatic hydrolysis reactor 150 may have a total solids concentration of 5 to 25 weight percent. The cellulosic biomass pulp, acid and enzyme suspension may stay within the enzymatic hydrolysis reactor 150 for any amount of time, preferably 12 to 100 hours. During this time, the enzyme transforms the solid materials to a quantity of monomeric sugar solution. The quantity of monomeric sugar solution may include both C5 and C6 sugars. When the transformation has reached a satisfactory point, the quantity of monomeric sugar solution is output via line 152 to the second filter 160, which filters the quantity of monomeric sugar solution to remove unconverted pulp, lignin and other residual solid matter. The second filter 160 may be any type of filter, such as but not limited to, a filter press, centrifuge or ultra-filtration unit, which is capable of substantially separating the solid matter from the monomeric sugar solution. The filtered residual solid matter may be removed via line 162. The filtered quantity of monomeric sugar solution may then be transferred into a water removal device 172 via line 164, which may remove a quantity of water via line 173, to concentrate the filtered quantity of monomeric sugar to approximately 100-800 g/l. The concentrated filtered quantity of monomeric sugar may then be processed within a electrodialysis unit 170, connected to the water removal device 172 via line 176 to remove salts, acids, heavy metals, and lignin which are removed from the sugar solution by transferring them to a brine solution which enters the device via line 177 and leaves via line 178, thereby providing a sugar solution. The quantity of sugar solution may then be output via line 180 as a product of the system 100.

It is noted that the water removal device 172 may be used optionally or used at any position within the system 100.

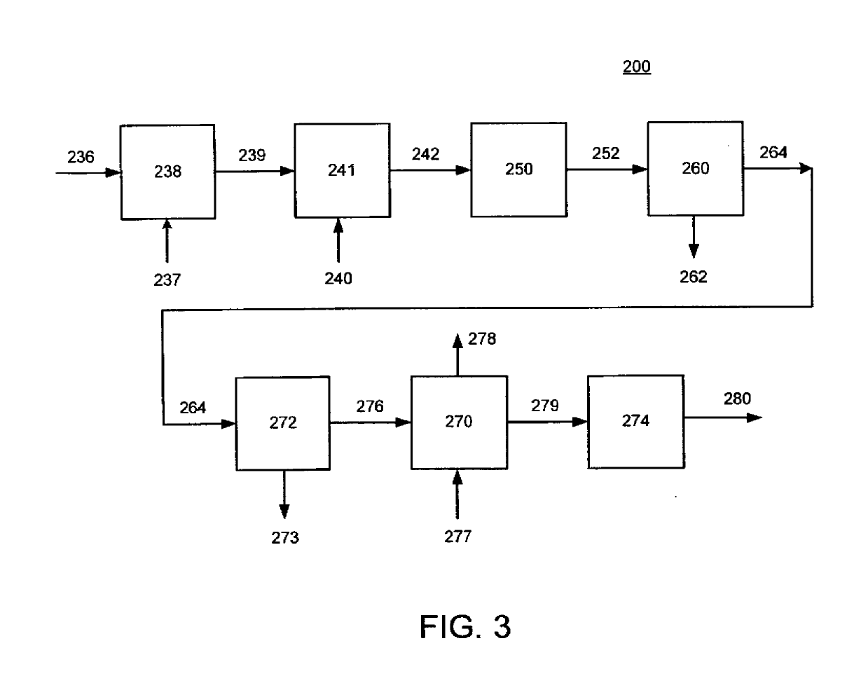

FIG. 3 is a schematic illustration of a system 200 for converting cellulose rich biomass pulp into a C6 rich sugar solution, in accordance with a third exemplary embodiment of the present disclosure. The system 200 for producing a C6 rich sugar solution, which may be referred to herein as ‘the system 200’, may include any of the features, components, or functions disclosed with respect to any of the other embodiments within this disclosure. The system 200 includes a cellulosic biomass pulp suspension within conduit 236 which is combined with a quantity of sulfuric acid via conduit 237 and then placed within a mixer 238. The mixed cellulosic biomass pulp and sulfuric acid are transported out of the mixer 238 via conduit 239 and combined with a quantity of enzyme that is transferred via conduit 240 into mixer 241 and then transferred by conduit 242 to an enzymatic hydrolysis reactor 250. The cellulosic biomass pulp, sulfuric acid, and the quantity of enzyme suspension are held within the enzymatic hydrolysis reactor 250 for a period of time, preferably between 12-100 hours although other period of time may be acceptable. While in the enzymatic hydrolysis reactor 250, the cellulosic biomass pulp, sulfuric acid, and the quantity of enzyme suspension are subjected to an approximate temperature between 35-70° C. at an approximate pH of 3.5-7.5. The cellulosic biomass pulp, sulfuric acid, and the quantity of enzyme suspension are output as a quantity of C6 rich monomeric sugar solution via output line 252, wherein the quantity of monomeric sugar solution may have a total sugar concentration of approximately between 5% and 25%.

A variety of different devices and components to the system 200 may be used at various stages. Commonly, after the enzymatic hydrolysis reactor 250, the quantity of C6 rich monomeric sugar solution is transferred to a filter 260 to remove unprocessed pulp and lignin, from the quantity of C6 rich monomeric sugar solution. The filter may be any type of filtering device that can separate suspended solids from liquids, such as but not limited to filter press, centrifuge or ultra-filtration system. Any residual solids may be output from the filter 260 via output 262. The filtered quantity of C6 rich monomeric sugar solution may then be transferred into a water removal device 272 via line 264, which may remove a quantity of water via line 273, to concentrate the filtered quantity of C6 rich monomeric sugar solution to approximately 100-800 g/l total sugar concentration. The concentrated filtered quantity of C6 rich monomeric sugar may then be processed within an electrodialysis unit 270, connected to the evaporator 272 via line 276 to remove salts, acids, heavy metals, and lignin which are removed from the sugar solution by transferring them to a brine solution which enters the device via line 277 and leaves via line 278. The C6 rich monomeric sugar solution leaves the electrodialysis unit 270 via line 278 and may enter an adsorption unit 274 via line 279 where further amounts of lignin, acids, salts and coloring matter may be removed. The adsorbents in this unit may be any of a variety of activated char and/or a variety of ion exchange resins depending on the specific applications to which the C6 rich monomeric sugar will be used. The C6 rich monomeric sugar solution is outputted as product via line 280.

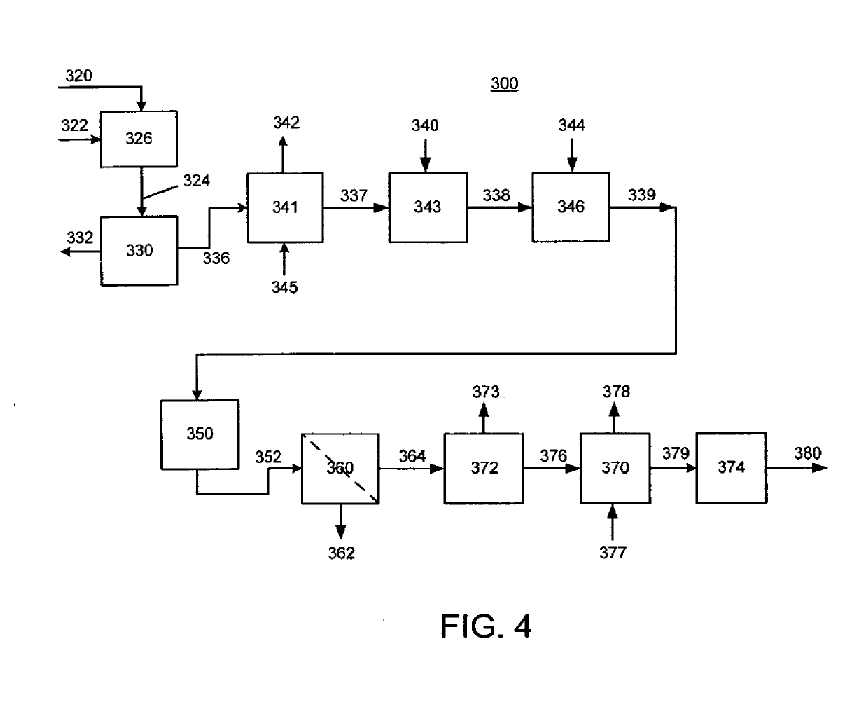

FIG. 4 is a schematic illustration of a system 300 for converting cellulose rich biomass into a C6 rich sugar solution, in accordance with a fourth exemplary embodiment of the present disclosure. The system 300 for converting cellulose rich biomass into C6 rich sugar, hereinafter simply referred to as the ‘system’ 300, may be used to produce a C6 rich sugar solution from a cellulose rich biomass feedstock, and may include any of the features, components, or functions disclosed with respect to any of the other embodiments within this disclosure.

As is shown in FIG. 4, an input line 320 may input a quantity of cellulose rich biomass into the system 300. The quantity of cellulose rich biomass may be transported into a vessel 326, which may be a traditional chemical pulp-cooking device, which utilizes heat and chemicals to separate a quantity of lignin from a quantity of fiber within the quantity of biomass. A quantity of fresh cooking liquor may also be input into the vessel 326 via line 322. The result is a quantity of cellulose rich biomass pulp suspension, created from the quantity of cellulose rich biomass, which leaves the vessel 326 via an outline line 324.

The process within the chemical pulp-cooking device may include any type of biomass pulping process, including kraft, sulfite, soda, or near neutral sulfite. For example, in the kraft process, the woody biomass is treated with a mixture of sodium hydroxide, sodium carbonate, and sodium sulfide, known as ‘white liquor’ to break the bonds that link the lignin to the cellulose. Any of the various chemical processes used may have specific parameters and requirements, any of which may be incorporated into the system 300 without reservation. It should also be noted that mechanical or thermal mechanical pulping processes may be used.

The quantity of cellulose rich biomass pulp may be characterized as a material that is approximately 70%-98% cellulose. Other quantities of the cellulose within the quantity of cellulose rich biomass may also be within the scope of the present disclosure. The remainder of the quantity of cellulose rich biomass pulp, i.e., 2%-30%, may be primarily a hemi-cellulose material. As is described further herein the quantity of cellulose material may be processed into glucose or other C6 sugars, whereas any portion of the quantity of hemi-cellulose material may be processed into xylose or other C5 sugars.

Filter 330 may filter the quantity of cellulose rich biomass pulp suspension to remove a quantity of cellulose rich biomass pulp suspension and a solution of spent cooking liquor, which is known as black liquor, and contains a quantity of the lignin which entered the process with the biomass. A portion of the cellulose rich biomass pulp suspension may be transferred to the next processing step through line 336. The black liquor or other spent cooking liquor may be disposed of or processed accordingly, such as via disposal line 332. The quantity of cellulose rich biomass pulp suspension is transferred by line 336 to a pulp washer 341 which intakes water via line 345 and outputs dilute spent cooking liquor via line 342. The cellulose rich biomass pulp suspension enters mixer 343 via line 337 where acid is added via line 340; the output continues through line 338 to mixer 346 where enzyme is added by line 344. The cellulose rich biomass, acid and enzyme suspension then passes to the enzyme hydrolysis reactor 350 through line 339. The enzymatic hydrolysis reactor may be any vessel or structure that allows for digestion, processing, or a catalytic transformation of the cellulose rich biomass and the acid and enzyme suspension to produce the quantity of C6 rich monomeric sugar solution. The enzymatic hydrolysis reactor 350 may have a predetermined temperature range and predetermined pH level to produce the quantity of monomeric sugar solution. For example, the enzymatic hydrolysis reactor 350 may have a temperature range of 35° C. to 70° C. and a pH level between 3.0 and 7.5, although other ranges are considered within the scope of the present disclosure. Additionally, the feed to the enzymatic hydrolysis reactor 350 may have a total solids concentration of 5 to 25 weight percent. The cellulose rich biomass pulp, acid, and enzyme suspension, may stay within the enzymatic hydrolysis reactor 350 for any amount of time, preferably 12 to 100 hours. During this time, the enzyme transforms the solid materials to a quantity of C6 rich monomeric sugar solution. The quantity of C6 rich monomeric sugar solution may include both C5 and C6 sugars. When the transformation has reached a satisfactory point, the quantity of C6 rich monomeric sugar solution is output via line 352 to the second filter 360, which filters the quantity of C6 rich monomeric sugar solution to remove residual solid matter. The second filter 360 may be any type of filter, such as but not limited to, a filter press, centrifuge or ultra-filtration unit, which is capable of substantially separating the solid matter from the C6 rich monomeric sugar solution. The filtered residual solid matter may be removed via line 362. The filtered quantity of C6 rich monomeric sugar solution may then be transferred into a water removal device 372 via line 364, which may remove a quantity of water via line 373, to concentrate the filtered quantity of C6 rich monomeric sugar to approximately 100-800 g/l. The concentrated filtered quantity of C6 rich monomeric sugar solution may then be processed within an electrodialysis unit 370, connected to the evaporator 372 via line 376 to remove salts, acids, heavy metals, and lignin which are removed from the sugar solution by transferring them to a brine solution which enters the device via line 377 and leaves via line 378. The C6 rich monomeric sugar solution leaves the electrodialysis unit 370 via line 379 and may enter an adsorption unit 374 where further amounts of lignin, acids, salts and coloring matter may be removed. The adsorbents in this unit may be any of a variety of activated char and/or a variety of ion exchange resins depending on the specific applications to which the C6 rich monomeric sugar will be used. The C6 rich monomeric sugar solution is outputted as product via line 380 as a product of the system 300. It is noted that the water removal device 372 may be used optionally or used at any position within the system 300.

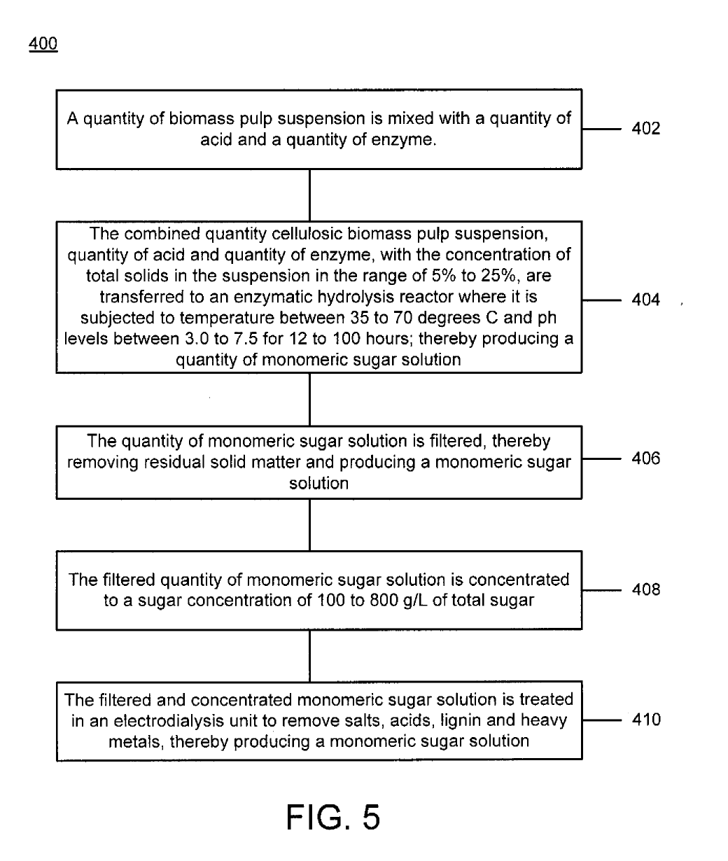

FIG. 5 is a flowchart 400 illustrating a process for converting cellulosic biomass pulp into a sugar solution, in accordance with the first exemplary embodiment of the present disclosure. It should be noted that any process descriptions or blocks in flow charts should be understood as representing modules, segments, or steps that include one or more instructions for implementing specific chemical or physical changes to the materials in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by block 402, a quantity of cellulosic biomass pulp suspension is mixed with a quantity of acid and a quantity of enzyme. The combined quantity of cellulosic biomass pulp suspension, quantity of acid and quantity of enzyme, with the concentration of total solids in the suspension in the range of 5% to 25%, are transferred to an enzymatic hydrolysis reactor where it is subjected to temperature between 35 to 70 degrees C. and ph levels between 3.0 to 7.5 for 12 to 100 hours; thereby producing a quantity of monomeric sugar solution (block 404). The quantity of monomeric sugar solution is filtered, thereby removing residual solid matter and producing a monomeric sugar solution (block 406). The filtered quantity of monomeric sugar solution is concentrated to a sugar concentration of 100 to 800 g/L of total sugar (block 408). The filtered and concentrated monomeric sugar solution is treated in an electrodialysis unit to remove salts, acids, lignin and heavy metals, thereby producing a monomeric sugar solution (block 410). Any additional steps or variations not explicitly discussed may also be included with the method, all of which are considered within the scope of the present disclosure.

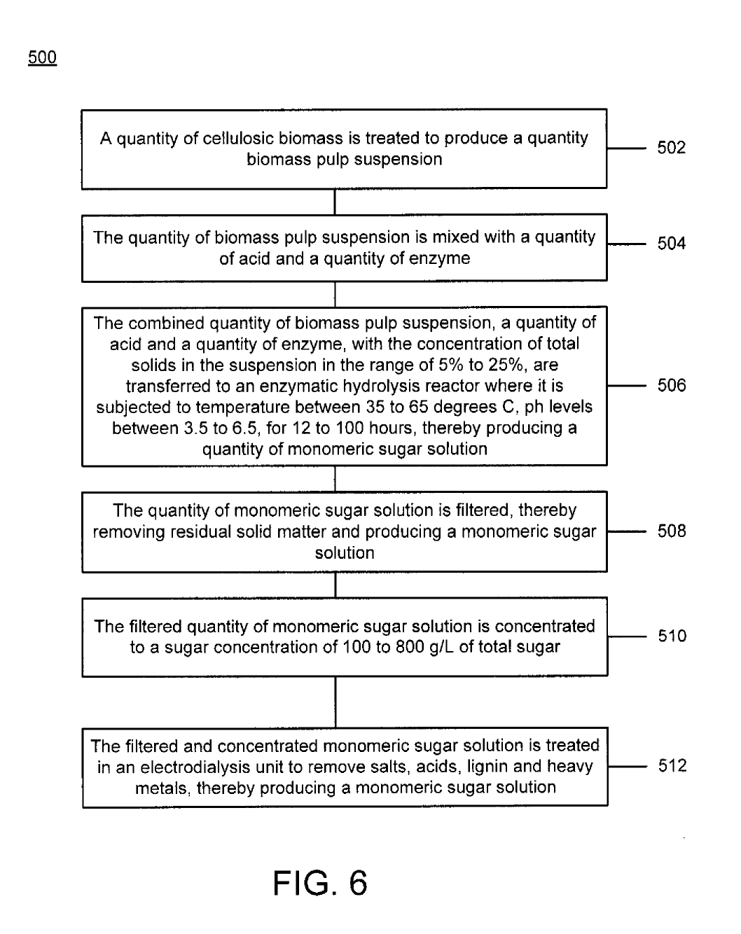

FIG. 6 is a flowchart 500 illustrating a process for converting cellulosic biomass into a sugar solution, in accordance with the second exemplary embodiment of the present disclosure. As is shown in block 502, a cellulosic biomass pulp is treated to produce a quantity of cellulosic biomass pulp suspension. The quantity of cellulosic biomass pulp suspension is mixed with a quantity of acid and a quantity of enzyme (block 504). The combined quantity of cellulosic biomass pulp suspension, a quantity of acid and a quantity of enzyme, with the concentration of total solids in the suspension in the range of 5% to 25%, are transferred to an enzymatic hydrolysis reactor where it is subjected to temperature between 35 to 65 degrees C., ph levels between 3.5 to 6.5, for 12 to 100 hours, thereby producing a quantity of monomeric sugar solution (block 506). The quantity of monomeric sugar solution is filtered, thereby removing residual solid matter and producing a monomeric sugar solution (block 508). The filtered quantity of monomeric sugar solution is concentrated to a sugar concentration of 100 to 800 g/L of total sugar (block 510). The filtered and concentrated monomeric sugar solution is treated in an electrodialysis unit to remove salts, acids, lignin and heavy metals, thereby producing a monomeric sugar solution (block 512). Any additional steps or variations not explicitly discussed may also be included with the method, all of which are considered within the scope of the present disclosure.

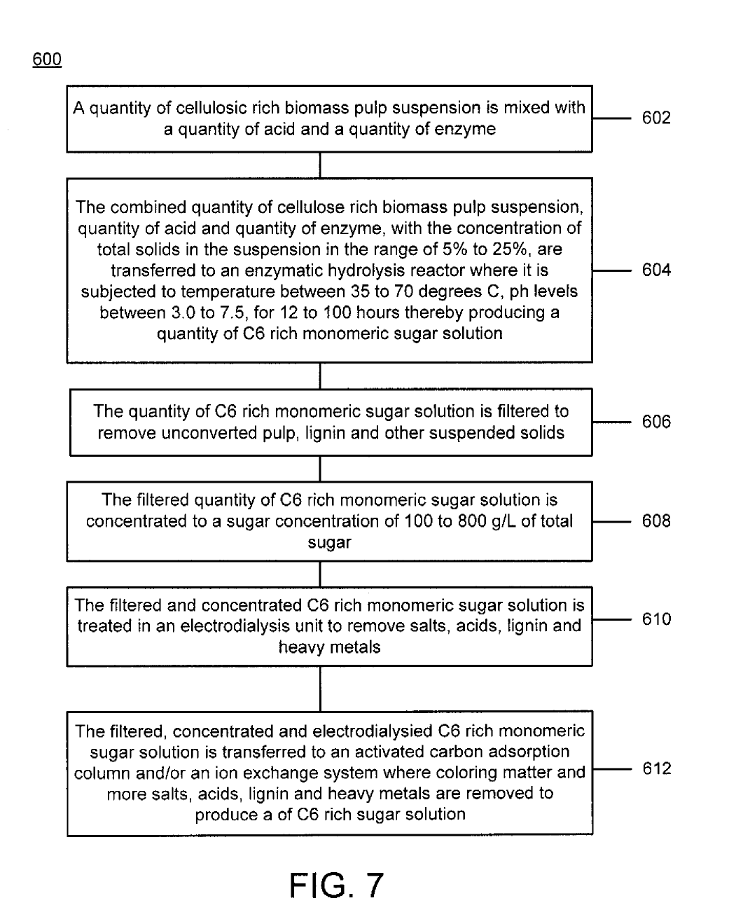

FIG. 7 is a flowchart 600 illustrating a process for converting cellulose rich biomass pulp into a C6 rich sugar solution, in accordance with the third exemplary embodiment of the present disclosure. As shown in block 602, a quantity of cellulosic rich biomass pulp suspension is mixed with a quantity of acid and a quantity of enzyme. The combined quantity of cellulose rich biomass pulp suspension, quantity of acid and quantity of enzyme, with the concentration of total solids in the suspension in the range of 5% to 25%, are transferred to an enzymatic hydrolysis reactor where it is subjected to temperature between 35 to 70 degrees C., ph levels between 3.0 to 7.5, for 12 to 100 hours thereby producing a quantity of C6 rich monomeric sugar solution (block 604). The quantity of C6 rich monomeric sugar solution is filtered to remove unconverted pulp, lignin and other suspended solids (block 606). The filtered quantity of C6 rich monomeric sugar solution is concentrated to a sugar concentration of 100 to 800 g/L of total sugar (block 608). The filtered and concentrated C6 rich monomeric sugar solution is treated in an electrodialysis unit to remove salts, acids, lignin and heavy metals (block 610). The filtered, concentrated and electrodialysied C6 rich monomeric sugar solution is transferred to an activated carbon adsorption column and/or an ion exchange system where coloring matter and more salts, acids, lignin and heavy metals are removed to produce a of C6 rich sugar solution (block 612). Any additional steps or variations not explicitly discussed may also be included with the method, all of which are considered within the scope of the present disclosure.

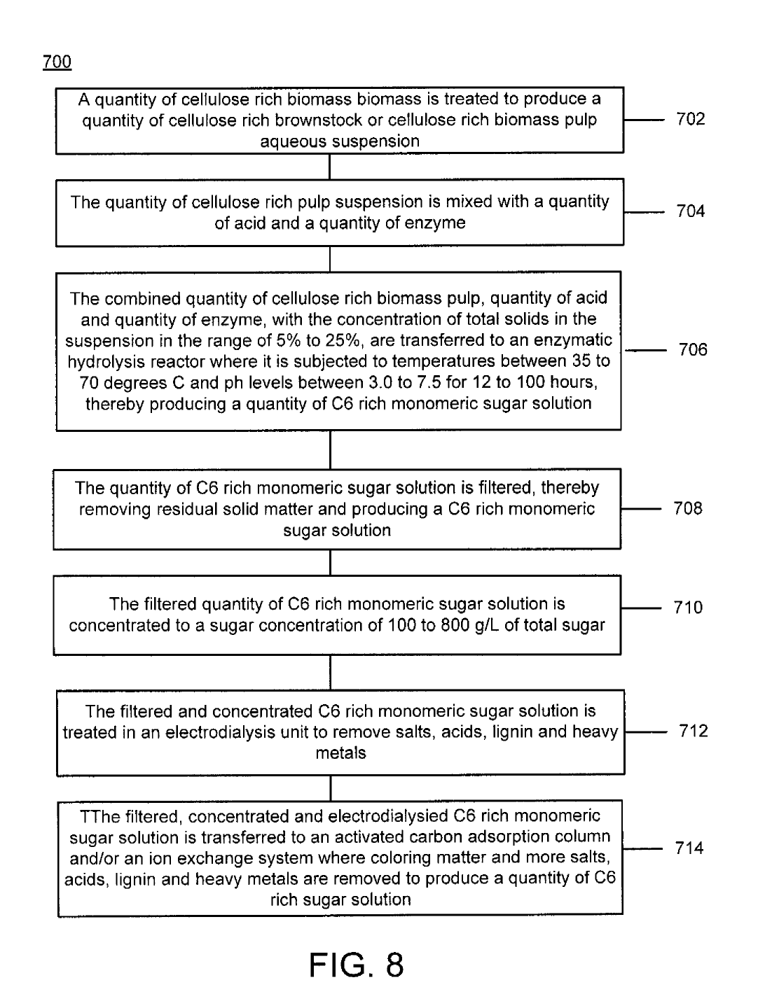

FIG. 8 is a flowchart 700 illustrating a process for converting cellulose rich biomass into a C6 rich sugar solution, in accordance with a fourth exemplary embodiment of the present disclosure. As shown in block 702, a quantity of cellulose rich biomass is treated to produce a quantity of cellulose rich biomass pulp suspension. The quantity of cellulose rich biomass pulp suspension is mixed with a quantity of acid and a quantity of enzyme (block 704). The combined quantity of cellulose rich biomass pulp, quantity of acid and quantity of enzyme suspension, with the concentration of total solids in the suspension in the range of 5% to 25%, are transferred to an enzymatic hydrolysis reactor where it is subjected to temperatures between 35 to 70 degrees C. and ph levels between 3.0 to 7.5 for 12 to 100 hours, thereby producing a quantity of C6 rich monomeric sugar solution (block 706). The quantity of C6 rich monomeric sugar solution is filtered, thereby removing residual solid matter and producing a C6 rich monomeric sugar solution (block 708). The filtered quantity of C6 rich monomeric sugar solution is concentrated to a sugar concentration of 100 to 800 g/L of total sugar (block 710). The filtered and concentrated C6 rich monomeric sugar solution is treated in an electrodialysis unit to remove salts, acids, lignin and heavy metals (block 712). The filtered, concentrated and electrodialysied C6 rich monomeric sugar solution is transferred to an activated carbon adsorption column and/or an ion exchange system where coloring matter and more salts, acids, lignin and heavy metals are removed to produce a quantity of C6 rich sugar solution (block 714). Any additional steps or variations not explicitly discussed may also be included with the method, all of which are considered within the scope of the present disclosure.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and the present disclosure and protected by the following claims.

1. A process for converting cellulosic biomass pulp suspension into a sugar solution, the process comprising the steps of:
combining a quantity of cellulosic biomass pulp suspension with a quantity of acid and a quantity of enzyme; and
placing the combined quantity of cellulosic biomass pulp, enzyme, and acid suspension in an enzymatic hydrolysis reactor having a predetermined temperature range and predetermined pH level, thereby producing a quantity of monomeric sugar solution.

2. The process of claim 1, further comprising the step of treating a quantity of cellulosic biomass to produce the quantity of cellulosic biomass pulp suspension.

3. The process of claim 2, wherein the step of treating the quantity of cellulosic biomass further comprises the step of:
separating a quantity of lignin and a quantity of fiber from the quantity of cellulosic biomass, thereby producing a quantity of cellulosic pulp; and
filtering and washing the quantity of cellulosic pulp to remove a quantity of spent cooking chemicals and to thereby producing the quantity of cellulosic biomass pulp suspension.

4. The process of claim 3, wherein the separating the quantity of lignin and the quantity of fiber from the quantity of cellulosic biomass further comprises a pulping process utilizing a combination of heat and at least one chemical within a chemical pulp-cooking vessel.

5. The process of claim 4, wherein the pulping process further comprises a chemical pulping process, wherein the chemical pulping process includes at least one process of kraft, sulfite, soda, and near neutral sulfite pulping process.

6. The process of claim 3, wherein the quantity of cellulosic pulp further comprises approximately 60%-98% cellulose.

7. The process of claim 1, further comprising the step of filtering the quantity of monomeric sugar solution, thereby removing residual solid matter and producing a filtered sugar solution.

8. The process of claim 7, further comprising the step of removing water from the filtered sugar solution to a concentration of approximately 100-800 g/l.

9. The process of claim 7, further comprising the step of treating the filtered sugar solution in an adsorption unit to remove residual salts, acids, lignin, heavy metals and coloring matter.

10. The process of claim 7, further comprising the removed residual solid matter further comprises residual solids that are not metabolized by the quantity of enzyme within the enzymatic hydrolysis reactor.

11. The process of claim 7, further comprising the step of removing a quantity of salt, acids, heavy metals, and lignin from the filtered sugar solution with an electrodialysis unit.

12. The process of claim 7, further comprising the steps of:
evaporating the filtered sugar solution to a concentration of approximately 100-800 g/l;
removing a quantity of salt, acids, heavy metals, and lignin from the filtered sugar solution with an electrodialysis unit; and
treating the filtered sugar solution in an adsorption unit, wherein the steps of evaporating and treating the filtered sugar solution further comprises removing remaining inhibitors from the filtered sugar solution, wherein the remaining inhibitors include at least one of a quality of salt, a quality of residual acid, a quantity of lignin, a quantity of heavy metal, and a quantity of coloring matter.

13. The process of claim 1, wherein the enzymatic hydrolysis reactor has a temperature range of 35° C. to 70° C. and a pH level between 3.0 and 7.5.

14. The process of claim 1, wherein the enzymatic hydrolysis reactor has a total solid fee between 5% and 25%.

15. The process of claim 1, wherein the combined quantity of cellulosic biomass pulp, enzyme, and acid suspension are within the enzymatic hydrolysis reactor for between 12 and 100 hours.

16. The process of claim 1, wherein the quantity of monomeric sugar solution includes both C5 and C6 sugars.

17. An apparatus for converting cellulosic biomass pulp into sugar comprising:
a first mixer for mixing cellulosic biomass pulp suspension and a quantity of acid;
a second mixer fed from the first mixer for mixing the cellulosic biomass pulp and acid with a quantity of enzyme; and
an enzymatic hydrolysis reactor receiving the mixed cellulosic biomass pulp, acid, and enzyme suspension, wherein the enzymatic hydrolysis reactor has a predetermined temperature range and a predetermined pH level, and wherein the enzymatic hydrolysis reactor outputs a quantity of monomeric sugar solution.

18. The apparatus of claim 17 further comprising a vessel receiving a quantity of cellulose biomass and outputting a quantity of cellulose biomass pulp with cooking liquor; and a separation unit fed from the vessel for separating the quantity of cellulose biomass pulp suspension from the cooking liquor, wherein the separating unit feeds the first mixer.

19. The apparatus of claim 17, further comprising a filter positioned to receive the monomeric sugar solution and filter out residual solid matter from the monomeric sugar solution.

20. The apparatus of claim 19, further comprising an adsorption unit connected to the filter and producing a quantity of C6 sugar rich solution from the filtered monomeric sugar solution.

21. The apparatus of claim 19, further comprising a water removal unit positioned to receive the quantity of sugar solution, whereby the water removal unit outputs the quantity of sugar solution with a concentration of approximately 100-800 g/l.

22. The apparatus of claim 19, further comprising an electrodialysis unit in fluid communication with at least one of the filter, the adsorption unit, and the water removal unit, wherein the electrodialysis unit removes a quantity of salts, acids, lignin or heavy metal from the filtered sugar solution.

23. The apparatus of claim 22, further comprising an adsorption unit connected to the electrodialysis unit, wherein the adsorption unit further removes the quantity of salts, acids, heavy metals, lignin and coloring matter, thereby producing the quantity of C6 rich sugar solution.

24. The apparatus of claim 17, wherein the enzymatic hydrolysis reactor has a total solid feed between 5% and 25%.

25. The apparatus of claim 17, wherein the enzymatic hydrolysis reactor has a temperature range of 35° C. to 70° C. and a pH level between 3.0 and 7.5.

26. A process for converting cellulose rich biomass pulp suspension into a C6 rich sugar solution, the process comprising the steps of:
combining a quantity of cellulose rich biomass pulp suspension with a quantity of acid and a quantity of enzyme; and
placing the combined quantity of cellulose rich biomass pulp, enzyme, and acid in an enzymatic hydrolysis reactor having a predetermined temperature range and predetermined pH level, thereby producing a quantity of monomeric C6 rich sugar solution.
